(19) United States
(12) Patent Application Publication
(10) Pub. No.: US 2006/0237716 A1
(43) Pub. Date: Oct. 26, 2006

(54) Material and cell structure for storage applications

(76) Inventors: Recai Sezi, Roettenbach (DE); Andreas Walter, Dresden (DE); Reimund Engl, Regensburg (DE); Anna Maltenberger, Leutenbach (DE); Joerg Schumann, Dresden (DE)

Correspondence Address: JENKINS, WILSON, TAYLOR & HUNT, P. A., 3100 TOWER BLVD, SUITE 1200, DURHAM, NC, 27707, US

(21) Appl. No.: 11/392,238

(22) Filed: Mar. 29, 2006

Related U.S. Application Data
(63) Continuation of application No. PCT/EP04/10924, filed on Sep. 30, 2004.

(30) Foreign Application Priority Data
Sep. 30, 2003 (DE) 10345403.9

(51) Int. Cl. H01L 29/08; H01L 51/00; C07D 409/04; H01L 21/20

(52) U.S. Cl. 257/40; 549/35; 438/584

(57) ABSTRACT

The present invention relates to compositions for storage applications, relates to a memory cell which comprises the abovementioned composition and two electrodes and furthermore relates to a process for the production of microelectronic components and the use of the composition according to the invention in the production of these microelectronic components.

### RELATED APPLICATIONS

This application is a continuation of PCT patent application number PCT/EP2004/010924, filed Sep. 30, 2004, which claims priority to German patent application number 10345403.9, filed Sep. 30, 2003, the disclosures of each of which are incorporated herein by reference in their entirety.

### TECHNICAL FIELD

The present invention relates to compositions for storage applications, relates to a memory cell which comprises the abovementioned composition and two electrodes and furthermore relates to a process for the production of microelectronic components and the use of the composition according to the invention in the production of these microelectronic components.

### BACKGROUND ART

The electronic and optoelectronic applications of organic semiconductors include light-emitting diodes, field effect transistors, apparatuses for switching memories, memory elements, logic elements and finally complex lasers. Because the industry is changing over from material—to molecule-based electronics, there is an increasing trend to consider in more detail the voltage-induced switching phenomena in conjugated organic compounds, which were observed for the first time more than 30 years ago.

Nonvolatile and simultaneously fast memories are the basic requirement for many portable devices, such as, for example, laptop, PDA, mobile telephone, digital cameras, HDTV devices, etc.; in such devices, no boot process should be required on switching on and a sudden power failure should not lead to a loss of data. In addition to materials having ferroelectric properties or memory elements consisting of magnetic tunnel junctions (MTJs), materials which can change their resistance reversibly between two stable states (resistive effect) are particularly suitable for a nonvolatile memory. The two different resistance values can be detected via the current flow. A further advantage of the resistive memory, for example compared with the memory with a ferroelectric effect, is that the memory state is not cleared on reading out and does not have to be rewritten. Compared with memory elements consisting of MTJs, which consist of a plurality of complex layer sequences, memory elements comprising resistive materials have a very simple structure.

In switching devices which can be used as memory elements, two differently conducting states are observed at the same applied voltage. The two differently conducting states are stable up to a certain magnitude of voltage and can be converted one into the other on exceeding these threshold voltages. The reversible switching back and forth between these two differently conducting states is generally effected by pole reversal of the voltage, it being necessary for the magnitude of the voltage to be somewhat greater than the respective threshold voltages. For the detection of the two differently conducting states, i.e. for the determination of the resistance, the applied voltage must be below the threshold voltage so that conversion into the other state is prevented. Several possible mechanisms were discussed for explaining the existence of the two states. The conducting states which were observed in thin anthracene films and in structures based on Cr-doped inorganic oxide films were attributed to the presence of traps which are filled under strong fields, which leads to a high charge carrier mobility via a filamentary state. In a complicated three-layer structure, an additional metal layer was introduced between two active organic layers in order to store charges and to provide switching with high conductivity (current ratio between the two states, ON:OFF ratio=10⁶). In these high-performance devices in which a switching mechanism is a bulky feature, miniaturization thereof to the molecular order of magnitude is limited.

In one-layer molecular switching devices, the ON-OFF ratio is generally low (50-80) and the memory lasts only minutes (about 15 minutes in nitroamine-based systems). The origin of the highly conducting state was attributed to the conjugation modification via an electroreduction of the molecules. The method for increasing the ON-OFF ratio consists in either increasing the current in the ON state or reducing the current in the OFF state. With the aim of generating a molecule having an OFF state of very low conductivity, Rose Bengal, which has electron acceptor groups distributed over the entire surface of the molecule, was chosen in the prior art. In the absence of donor groups, the density of the electron distribution in the benzene rings is reduced and the conjugation in the molecule is greatly influenced.

The publication “Large conductance switching and memory effects in organic molecules for data-storage applications”, A. Bandyopadhyay et al., Applied Physics Letters, vol. 82, No. 8, 24 Feb. 2003, reports on switching with conductivity in Rose Bengal with a high ON-OFF ratio by restoring the conjugation of the molecules. Memory effects were also described in devices which enable these structures to operate in data-storage applications. With the devices disclosed there, it was possible to write or to clear the state and to read this for many cycles. In switching devices, the active semiconductor maintained its conducting state until a blocking voltage cleared said state. A highly conducting state resulted owing to the restoration of the conjugation in the molecule via electroreduction. Such a high ON-OFF ratio in a one-layer sandwich structure is, in comparison with contemporary switching devices, attributable to a low creep or leakage current in the OFF state. The concept of restoration of conjugation was verified in supramolecular structures by addition of donor groups to the molecule, which resulted in an increased current in the OFF state and therefore a lower ON-OFF ratio. The abovementioned publication shows several generalized examples of the choice of organic molecules for achieving a high ON-OFF ratio in the molecular switching devices.

### SUMMARY OF THE INVENTION

In the light of this, it is the object of the present invention to provide a material which is switchable between two stable states of different resistivity and can therefore serve as a nonvolatile memory. It is a further object of the present invention to provide a material which serves for the abovementioned purposes and can be processed by customary methods in microelectronics, such as, for example, spin coating, and is switchable by means of the use of electrodes which are used in microelectronics. It is a further object of the present invention to provide an organic material as a nonvolatile memory, the material switching at low voltages.

These objects are achieved by the subject matter of the independent claims.

Preferred embodiments are evident from the subclaims.

As discussed above, it is in principle known that organic materials can serve as nonvolatile memories. In the abovementioned publication by A. Bandyopadhyay et al. (Applied Physics Letters, volume 82, No. 8, Feb. 24, 2003), however a material is described which requires very inconvenient processing (oven treatment for several hours in vacua) and is moreover reliant on an indium tin oxide electrode and switches only at voltages ≧3 V (cf. for example FIG. 5 of A. Bandyopadhyay et al.).

Accordingly, the material according to the invention has the particular advantage that it is switchable at voltages as low as ≦1 V.

The present invention achieves this by providing a novel material for storage applications which comprises a monomer M1 and additionally a monomer M2 and/or M3.

The present invention is directed in particular at the following aspects and embodiments:

According to a first aspect, the present invention relates to a composition for storage applications which comprises the following constituents:
a) a monomer M1, represented by the following formula 1 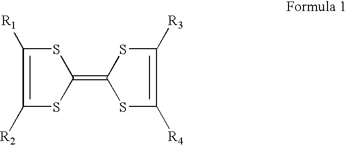
   in which R₁, R₂, R₃ and R₄, independently of one another, are H, F, Cl, Br, I, OH, SH, substituted or unsubstituted alkyl, alkenyl, alkynyl, O-alkyl, O-alkenyl, O-alkynyl, S-alkyl, S-alkenyl, S-alkynyl, aryl, heteroaryl, O-aryl, S-aryl, O-heteroaryl or S-heteroaryl, —(CF₂)ₙ—CF₃, —CF((CF₂)ₙCF₃)₂, -Q-(CF₂)ₙ—CF₃, —CF(CF₃)₂ or —C(CF₃)₂ or —C(CF₃)₃; and

n=from 0 to 10;
b) a monomer M2 and/or M3, represented by the following formulae 2 and 3: 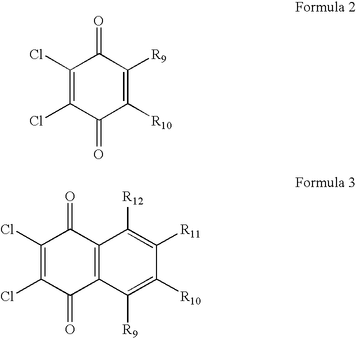
   in which R₉, R₁₀, R₁₁ and R₁₂, independently of one another, are F, Cl, Br, I, CN, NO₂, substituted or unsubstituted alkyl, alkenyl, alkynyl, O-alkyl, O-alkenyl, O-alkynyl, S-alkyl, S-alkenyl, S-alkynyl, aryl, heteroaryl, O-aryl, S-aryl, O-heteroaryl, S-heteroaryl, aralkyl or arylcarbonyl;
   in which Q is —O— or —S—.

According to the invention, the combinations of the monomers M1 and M2, M1 and M3 or M1, M2 and M3 are therefore possible.

According to a preferred embodiment, in formula 1, R₁, R₂, R₃ and R₄, independently of one another, are substituted or unsubstituted alkyl, O-alkyl, S-alkyl, aryl, heteroaryl, O-aryl, S-aryl, O-heteroaryl or S-heteroaryl.

In formulae 2 and/or 3, R₉, R₁₀, R₁₁ and R₁₂, independently of one another, are preferably Cl, CN or NO₂.

R₉, R₁₀, R₁₁ and R₁₂ in formulae 2 and/or 3, independently of one another, are particularly preferably

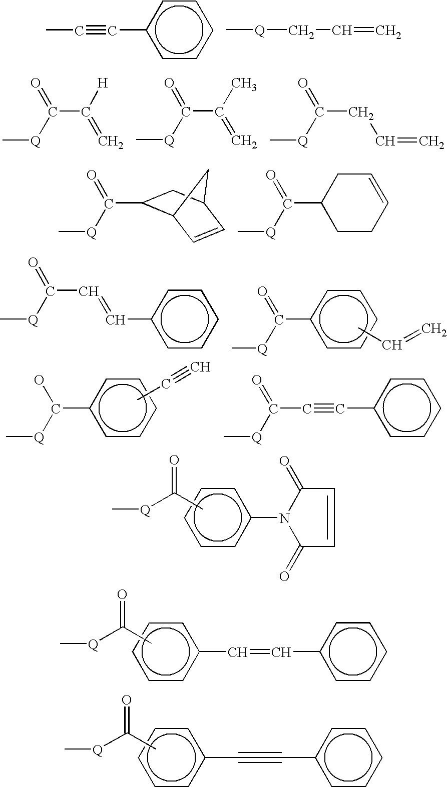

Tetrathiofulvalene (R₁-R₄═H) is a particularly preferred monomer for M1 and chloranil (R₉ and R₁₀═Cl) is a particularly preferred monomer for M2.

The term “alkyl” as used herein includes straight-chain and branched alkyl groups as well as cycloalkyl groups having 1-10, particularly preferably 1-6, carbon atoms. The terms “alkenyl, alkynyl” as used herein likewise relate to straight-chain and branched alkenyl and alkynyl groups, respectively, which have 1-10, particularly preferably 1-6, carbon atoms.

The term “aryl” as used herein relates to and includes aromatic hydrocarbon radicals preferably having 6-18, particularly preferably 6-10 carbon atoms.

According to a particularly preferred embodiment, the composition according to the invention furthermore comprises a polymer material. The monomers M1, M2 and/or M3 are formulated with said polymer material in a common, suitable solvent and this formulation is then further processed without problems, for example by means of spin coating.

Preferred polymer materials here are polyethers, polyethersulphones, polyether sulphides, polyether ketones, polyquinolines, polyquinoxalines, polybenzoxazoles, polybenzimidazoles, polymethacrylates or polyimides, including the precursors thereof and mixtures and copolymers thereof.

As mentioned at the outset, the mixture is preferably dissolved in a solvent. This solvent is preferably selected from N-methylpyrrolidone, gamma-butyrolactone, methoxypropyl acetate, ethoxyethyl acetate, ethers of ethylene glycol, in particular diethylene glycol diethyl ether, ethoxyethyl propionate and ethyl acetate.

As an alternative to the provision and subsequent mixing of the monomers M1, M2 and/or M3, these monomers can be chemically bonded to the polymer and then dissolved in a solvent.

According to a second aspect, the present invention is directed at a memory cell comprising a composition as defined above and two electrodes, the composition being arranged between the two electrodes.

Suitable electrodes are all materials customary in microelectronics, but in particular electrodes comprising AlSi, AlSiCu, copper, aluminium, titanium, tantalum, titanium nitride and tantalum nitride.

Here, the electrodes are preferably structured, the structuring preferably being effected by means of shadow masks or photolithographic techniques.

The layer thicknesses for the composition and the electrodes are preferably in each case from 20 nm to 2000 nm, particularly preferably from 50 nm to 200 nm.

By using adhesion promoters, the adhesion of the polymers to surfaces relevant in microelectronics, such as, for example, silicon, silicon oxide, silicon nitride, tantalum nitride, tantalum, copper, aluminium, titanium or titanium nitride, can be improved.

The following compounds can preferably be used as adhesion promoters:

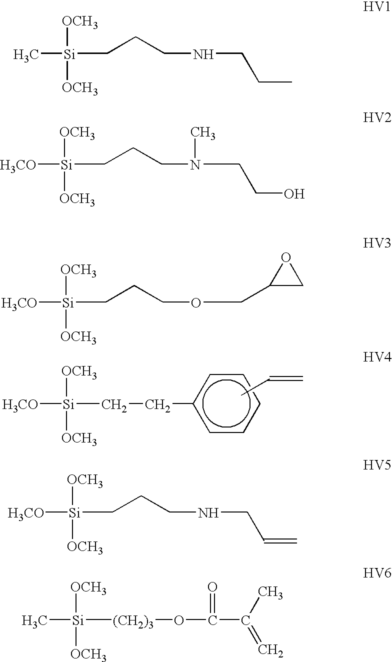

According to a further embodiment, the memory cell is present in combination with a diode, PIN diode or Z-diode or a transistor.

According to a third aspect, the invention is directed at a process for the production of microelectronic components, which comprises the following steps:
a) application of a first electrode to a silicon wafer,
b) application of a composition as defined herein to the electrode formed in a),
c) application of a second electrode to the layer formed in b).

According to a preferred embodiment, the application in steps a) and c) is effected by means of vapour deposition or sputtering.

Preferably, the composition in step b) is applied by spin coating and then dried.

According to a further preferred embodiment, the monomers present in the composition are applied simultaneously or directly in succession by means of vacuum vapour deposition. The composition according to the invention is preferably used in the production of microelectronic components or as a memory medium.

The present invention is explained in more detail below by the attached drawings and examples, there being no intention to limit the invention thereby.

### BRIEF DESCRIPTION OF THE DRAWINGS

FIG. 1 shows the exemplary cell structure of a memory cell according to the invention, comprising a silicon substrate having an SiO₂ surface, a layer of copper (sputtered) and, as the top layer, the materials according to the invention and titanium pads.

FIG. 2 shows the circuit diagram used for measuring the I(U) characteristic of the memory cell according to the invention. The SourceMeter Series 2400 from Keithley was used for the measurement.

FIG. 3 shows the typical I(U) characteristic of the cells according to the invention.

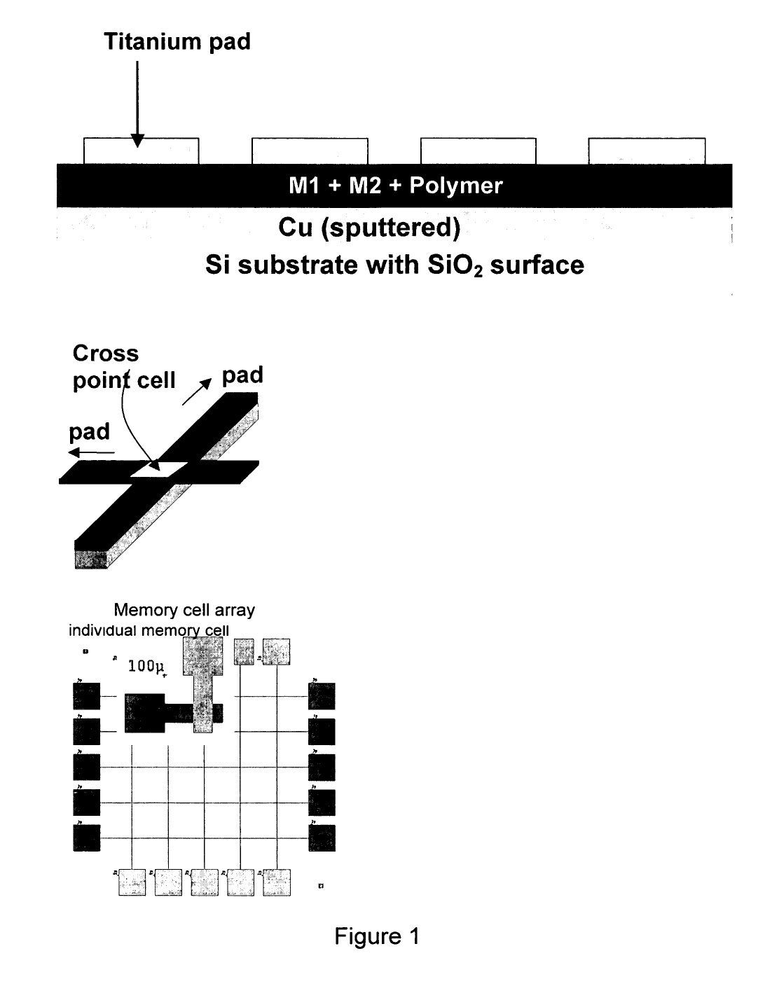

### DETAILED DESCRIPTION OF THE INVENTION

### EXAMPLES

### Example 1

### Production of the Bottom Electrode

The metal of the bottom electrode is applied to a silicon wafer having an insulating SiO or SiN surface by a vapour deposition method in a high vacuum or by a sputtering method. Metals which may be used are all metals relevant in microelectronics, such as, for example, copper, aluminium, gold, titanium, tantalum, tungsten, titanium nitride or tantalum nitride. The structuring of the metals can be effected either by application of the metals by means of shadow masks or by lithographic structuring with subsequent etching, by known methods, of the metals applied over the total surface.

### Example 2

### Preparation of Polymer Solutions

25 g of polyether, polyethersulphone, polyether ketone, polyimide, polybenzoxazole, polybenzimidazole or polymethacrylate are dissolved with 5 g of tetrathiafulvalene, and 5.98 g of chloranil in 75 g of distilled N-methylpyrrolidone (VLSI-Selectipur®) or distilled γ-butyrolactone (VLSI-Selectipur®). The dissolution process is expediently effected on a shaking apparatus at room temperature. The solution is then filtered under pressure through a 0.2 μm filter into a cleaned, particle-free sample tube. The viscosity of the polymer solution can be changed by varying the dissolved mass of polymer.

### Example 3

### Preparation of Polymer Solutions

25 g of polyether, polyethersulphone, polyether ketone, polyimide, polybenzoxazole, polybenzimidazole or polymethacrylate are dissolved with 4 g of tetrathiafulvalene, and 4.78 g of chloranil in 75 g of distilled N-methylpyrrolidone (VLSI-Selectipur®) or distilled γ-butyrolactone (VLSI-Selectipur®). The dissolution process is expediently effected on a shaking apparatus at room temperature. The solution is then filtered under pressure through a 0.2 μm filter into a cleaned, particle-free sample tube. The viscosity of the polymer solution can be changed by varying the dissolved mass of polymer.

### Example 4

### Preparation of Polymer Solutions

25 g of polyether, polyethersulphone, polyether ketone, polyimide, polybenzoxazole, polybenzimidazole or polymethacrylate are dissolved with 5 g of tetramethyl tetrathiafulvalene, and 4.35 g of dichlorodicyano-p-benzoquinone in 75 g of distilled N-methylpyrrolidone (VLSI-Selectipur®) or distilled γ-butyrolactone (VLSI-Selectipur®). The dissolution process is expediently effected on a shaking apparatus at room temperature. The solution is then filtered under pressure through a 0.2 μm filter into a cleaned, particle-free sample tube. The viscosity of the polymer solution can be changed by varying the dissolved mass of polymer.

### Example 5

### Improvement of the Adhesion by Adhesion Promoter Solutions

By using adhesion promoters, the adhesion of the polymers to surfaces relevant in microelectronics, such as, for example, silicon, silicon oxide, silicon nitride, tantalum nitride, tantalum, copper, aluminium, titanium or titanium nitride, can be improved.

For example, the following compounds can be used as adhesion promoters:

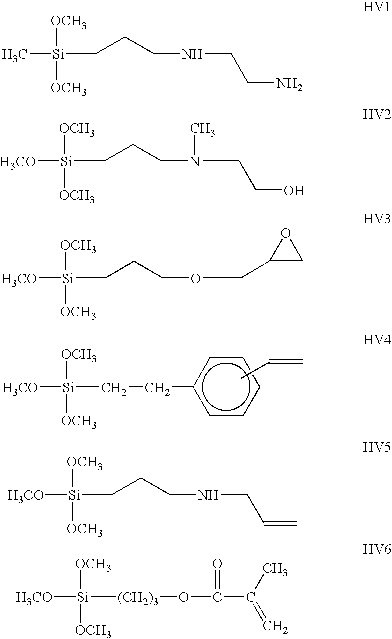

0.5 g of adhesion promoter (e.g. N-(2-aminoethyl)-3-aminopropylmethyldimethoxysilane) is dissolved in 95 g of methanol, ethanol or isopropanol (VLSI-Selectipur®) and 5 g of demineralized water in a cleaned, particle-free sample tube at room temperature. After standing for 24 h at room temperature, the adhesion promoter solution is ready for use. This solution can be used for up to 3 weeks. The adhesion promoter is intended to provide a monomolecular layer on the surface. The adhesion promoter can expediently be applied by the spin coating technique. For this purpose, the adhesion promoter solution is applied via a 0.2 μm prefilter and spun for 30 s at 5000 rpm. A drying step for 60 s at 100° C. is then effected.

### Example 6

### Application of a Polymer by the Spin Coating Method

The filtered solution of the polymer according to examples 2 to 4 is applied by means of a syringe to the silicon wafer processed according to example 1 or possibly the processed silicon wafer pretreated according to example 5 and distributed uniformly by means of a spin coater. The layer thickness should be in the range of 50-500 nm. Thereafter, the polymer is heated on a hotplate for 1 min at 120° C. and for 4 min at 200° C.

### Example 7

### Vapour Deposition of the Active Components

In addition to the method for applying the dissolved active components (donor and acceptor) in a polymer by spin coating, the components M1 and M2 or M3 can also be applied by the generally known method of vapour codeposition. The two components M1 and M2 are applied to the silicon wafer processed according to example 1, as far as possible in a molar ratio of 1:1, up to a layer thickness of 10-300 nm by vapour codeposition. The wafer should be cooled to 10-30° C.

### Example 8

### Production of the Top Electrode by Means of a Shadow Mask

The metal of the top electrode is applied by means of a shadow mask to the silicon wafer processed according to example 6 or 7 by a vapour deposition method in a high vacuum or by a sputtering method. Metals which may be used are all metals relevant in microelectronics, such as, for example, copper, aluminium, gold, titanium, tantalum, tungsten, titanium nitride or tantalum nitride.

### Example 9

### Production of the Top Electrode by a Lithographic Process

The metal of the top electrode is applied to the silicon wafer processed according to example 6 or 7 by a vapour deposition method in a high vacuum or by a sputtering method over the total surface. Metals which may be used are all metals relevant in microelectronics, such as, for example, copper, aluminium, gold, titanium, tantalum, tungsten, titanium nitride or tantalum nitride. For structuring the top electrode, a photoresist is applied to the metal by a spin-on method, exposed and structured. The metal not covered by the photoresist is then removed by etching by a known method. The photoresist still present is removed using a suitable stripper.

### Example 10

### Production of the Top Electrode by a Lift-Off Method

A photoresist is applied by a known method to the silicon wafer processed according to example 6 or 7 and is exposed and structured. The metal of the top electrode is then applied by a vapour deposition method in a high vacuum or by a sputtering method over the total surface. Metals which may be used are all metals relevant in microelectronics, such as, for example, copper, aluminium, gold, titanium, tantalum, tungsten, titanium nitride or tantalum nitride. By means of a lift-off process, the photoresist and the metal adhering to it are removed.

### Example 11

### Measurement of I(U) Characteristic

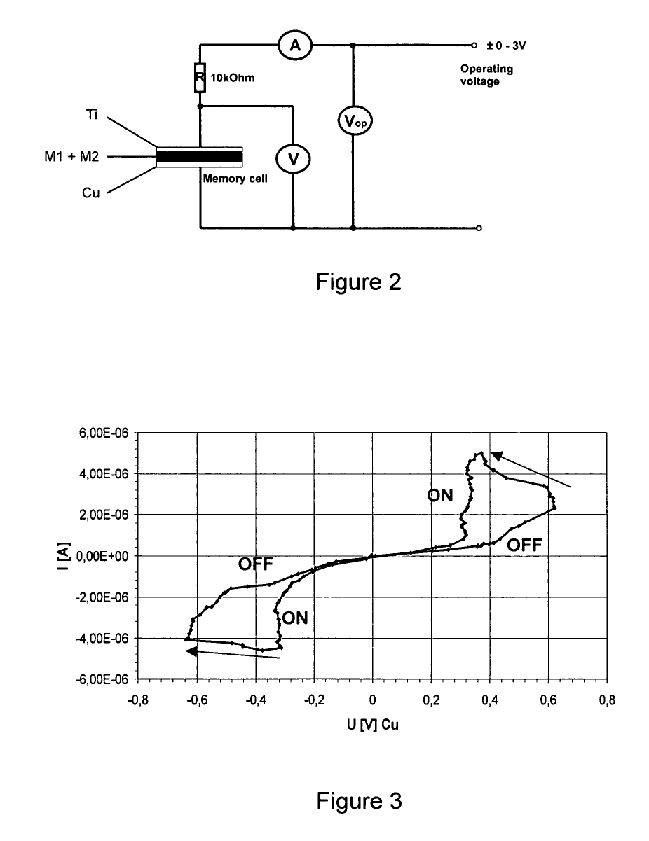

The measurement of the I(U) characteristic is effected according to the circuit diagram shown in FIG. 2.

For the measurement, the SourceMeter Series 2400 from Keithley was used. The cells exhibit the typical I(U) characteristic shown in FIG. 3.

The cells switch from a high-impedance state to a stable low-impedance state at about +0.6 V at Cu and back to a stable high-impedance state at −0.3 V at Cu. These two different resistance states are also stable in the voltage-free case.

1. Memory cell, comprising a composition defined below and two electrodes, the composition being arranged between the two electrodes, and wherein the composition comprises a polymer material and the following constituents:
a) a monomer M1, represented by the following formula 1 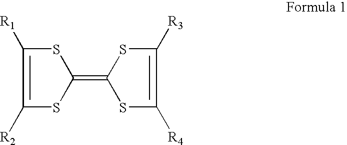
in which R₁, R₂, R₃ and R₄, independently of one another, are H, F, Cl, Br, I, OH, SH, substituted or unsubstituted alkyl, alkenyl, alkynyl, O-alkyl, O-alkenyl, O-alkynyl, S-alkyl, S-alkenyl, S-alkynyl, aryl, heteroaryl, O-aryl, S-aryl, O-heteroaryl or S-heteroaryl, —(CF₂)ₙ—CF₃, —CF((CF₂)ₙCF₃)₂, -Q-(CF₂)ₙ—CF₃, —CF(CF₃)₂ or —C(CF₃)₃; and
n=from 0 to 10;
b) a monomer M2 and/or M3, represented by the following formulae 2 and 3: 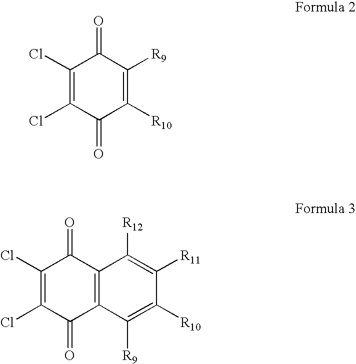
in which R₉, R₁₀, R₁₁ and R₁₂, independently of one another, are F, Cl, Br, I, CN, NO₂, substituted or unsubstituted alkyl, alkenyl, alkynyl, O-alkyl, O-alkenyl, O-alkynyl, S-alkyl, S-alkenyl, S-alkynyl, aryl, heteroaryl, O-aryl, S-aryl, O-heteroaryl, S-heteroaryl, aralkyl or arylcarbonyl;
in which Q is —O— or —S—.

2. Memory cell according to claim 1, in formula 1 R₁, R₂, R₃ and R₄, independently of one another, being substituted or unsubstituted alkyl, O-alkyl, S-alkyl, aryl, heteroaryl, O-aryl, S-aryl, O-heteroaryl or S-heteroaryl.

3. Memory cell according to claim 1, in formulae 2 and/or 3 R₉, R₁₀, R₁₁ and R₁₂, independently of one another, being Cl, CN or NO₂.

4. Memory cell according to claim 1, R₉, R₁₀, R₁₁ and R₁₂ in formulae 2 and/or 3, independently of one another, being

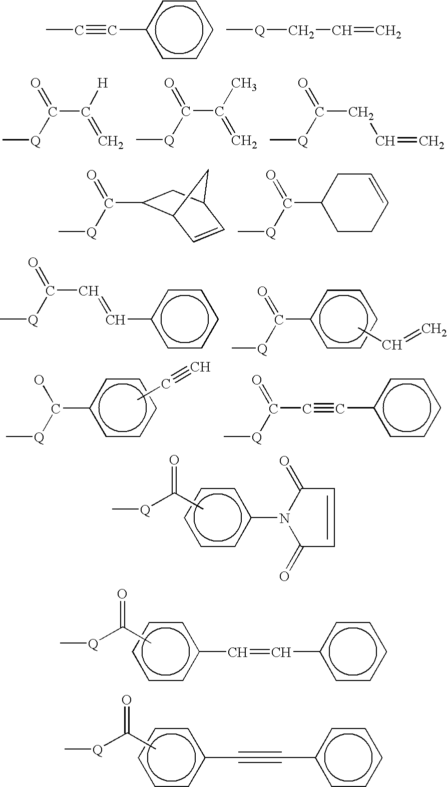

5. Memory cell according to claim 1, M1 being tetrathiofulvalene and M2 being chloranil.

6. Memory cell according to claim 1, the polymer material being selected from polyethers, polyethersulphones, polyether sulphides, polyether ketones, polyquinolines, polyquinoxalines, polybenzoxazoles, polybenzimidazoles, polymethacrylates or polyimides, including precursors thereof, and mixtures and copolymers thereof.

7. Memory cell according to claim 1, which furthermore comprises a solvent.

8. Memory cell according to claim 7, the solvent being selected from N-methylpyrrolidone, gamma-butyrolactone, methoxypropyl acetate, ethoxyethyl acetate, ethers of ethylene glycol, in particular diethylene glycol diethyl ether, ethoxyethyl propionate and ethyl acetate.

9. Memory cell according to claim 6, the monomers M1, M2 and/or M3 being chemically bonded to the polymer.

10. Memory cell according to claim 1, the electrodes being selected from AlSi, AlSiCu, copper, aluminium, titanium, tantalum, titanium nitride and tantalum nitride and combinations thereof.

11. Memory cell according to claim 10, the electrodes being structured.

12. Memory cell according to claim 11, the structuring being effected by means of shadow masks or photolithographic techniques.

13. Memory cell according to claim 1, the layer thicknesses for the composition and the electrodes being in each case from 20 nm to 2000 nm.

14. Memory cell according to claim 13, the layer thicknesses being in each case from 50 nm to 200 nm.

15. Memory cell according to claim 1, adhesion promoters for improving adhesion of the polymers to the relevant surfaces being used.

16. Memory cell according to claim 15, the adhesion promoter comprising one of the following compounds:

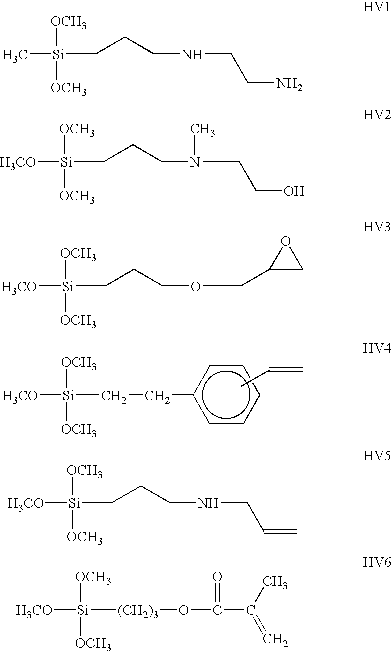

17. Memory cell according to claim 1, which is present in combination with a diode, PIN-diode, Z-diode or a transistor.

18. Process for the production of microelectronic components, which comprises the following steps:
a) applying of a first electrode to a silicon wafer,
b) applying of a composition according to claim 1 to the electrode formed in a),
c) applying of a second electrode to the layer formed in b).

19. Process according to claim 18, the application in steps a) and c) being effected by means of vapour deposition or sputtering.

20. Process according to claim 18, the composition in step b) being applied by spin coating and then dried.

21. Process according to claim 18, the monomers present in the composition being applied simultaneously or directly in succession by means of vacuum vapour deposition.

22. Use of a composition defined in claim 1 in the production of microelectronic components.

23. Use of the composition defined in claim 1 as a memory and switch medium.
